(19) United States
(12) Patent Application Publication
(10) Pub. No.: US 2006/0258877 A1
(43) Pub. Date: Nov. 16, 2006

(54) Process for the preparation of (E)-2-cyano-3-(3, 4-dihydroxy-5-nitrophenyl)-N, N-diethyl-2-propenamide (entacapone)

(75) Inventors: Pandurang Balwant Deshpande, Vadodara (IN); Parven Kumar Luthra, Vadodara (IN); Anand Kumar Pandey, Vadodara (IN); Dharmesh Ramniklal Dhameliya, Vadodara (IN)

Correspondence Address: HAMRE, SCHUMANN, MUELLER & LARSON, P.C., P.O. BOX 2902, MINNEAPOLIS, MN, 55402-0902, US

(73) Assignee: ALEMBIC LIMITED, Vadodara (IN)

(21) Appl. No.: 11/448,481

(22) Filed: Jun. 7, 2006

(30) Foreign Application Priority Data
Apr. 3, 2006 (IN) 509/MUM/2006

(51) Int. Cl. C07C 253/30

(52) U.S. Cl. 558/371

(57) ABSTRACT

The present invention relates to an improved process for the preparation of (E)-N,N-diethyl-2-cyano-3-(3,4-dihydroxy-5-nitrophenyl)acrylamide formula (I) 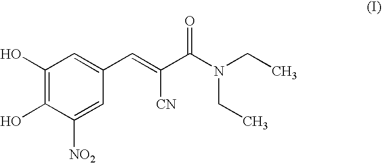 comprising steps of,
(a) condensation of 3,4-dihydroxy-5-nitrobenzaldehyde of formula (II) 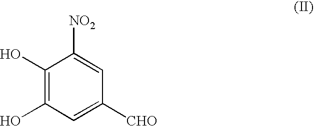
with N,N-diethylcyanoacetamide of formula (III) 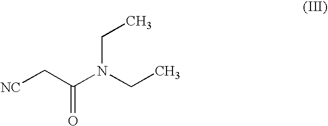
in the presence of a catalyst and optionally in the presence of phase transfer catalyst in a solvent selected from the group comprising of ethylacetate, acetonitrile, hydrocarbon such as toluene, xylene and like or mixture thereof to give mixture of (E) and (Z)-isomer of N,N-diethyl-2-cyano-3-(3,4-dihydroxy-5-nitrophenyl)acrylamide of formula (IV). 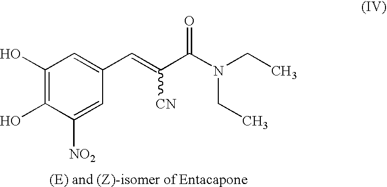 b) treating an isomeric mixture of (E) and (Z)-isomer of N,N-diethyl-2-cyano-3-(3,4-dihydroxy-5-nitrophenyl)acrylamide of formula (IV) obtained in step (a) with a halogen in catalytic amounts, in a solvent to give (E)-N,N-diethyl-2-cyano-3-(3,4-dihydroxy-5-nitrophenyl)acrylamide formula (I).

### FIELD OF THE INVENTION

The present invention relates to an improved process for the preparation of (E)-2-cyano-3-(3,4-dihydroxy-5-nitrophenyl)-N,N-diethyl-2-propenamide (Entacapone) of formula (I)

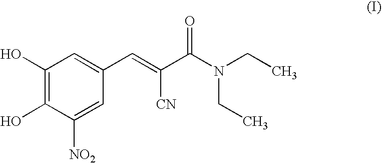

### BACKGROUND OF THE INVENTION

The chemical name of Entacapone is (E)-2-cyano-3-(3,4-dihydroxy-5-nitrophenyl)-N,N-diethyl-2-propenamide and molecular formula is C₁₄H₁₅N₃O₅ and molecular weight is 305.29. Entacapone is marketed by Orion Corporation under tradename Comtan® and is indicated for the treatment of Parkinson's disease.

(E)-2-cyano-3-(3,4-dihydroxy-5-nitrophenyl)-N,N-diethyl-2-propenamide (Entacapone) is a potent and specific peripheral catechol-O-methyltransferase (COMT) inhibitor. It is used in combination with levodopa/carbidopa to treat Parkinson's disease, sometime referred to as shaking palsy. Entacapone enhances the effect of levedopa/carbidopa by improving muscle control.

U.S. Pat. No. 4,963,590 describes a process for the preparation of crude N,N-diethyl-2-cyano-3-(3,4-dihydroxy-5-nitrophenyl)acrylamide of formula (IV). The synthetic process disclosed, comprises the condensation of 3,4-dihydroxy-5-nitrobenzaldehyde of formula (II) and N,N-diethylcyanoacetamide of formula (III) in anhydrous ethanol as shown below in Scheme-I

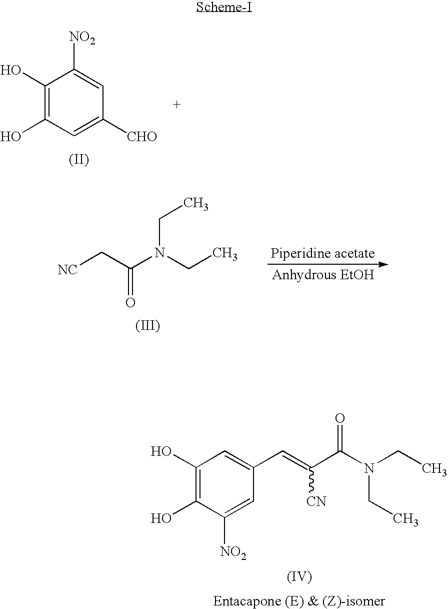

In the above process piperidine acetate was used as catalyst. Entacapone thus synthesized was obtained in 73% yield having a mixture of two geometrical isomeric forms, i.e., (E)- and (Z). There is no enabling disclosure for the preparation of (E)-isomer from the mixture of (E)- and (Z)-isomers of Entacapone in this patent.

The marketed form of Entacapone is E-isomer. Therefore, it is essentially to prepare (E)-isomer substantially free from (Z)-isomer.

Subsequently it is described in the U.S. Pat. No. 5,135,950 about preparing E-isomer and polymorphism-A from the mixture obtained from the reaction is reported in the GB patent No 2200109. It also discloses about the (E)- and (Z)-isomers having the structural formula: 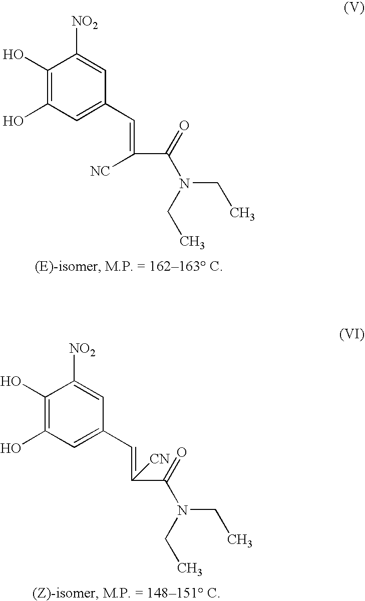
are obtained as mixture in the ratio of about 70-80% to about 30-20%, respectively.

U.S. Pat. No. 5,135,950 discloses that “crystallographically essentially pure” and stable polymorphic form A of (E)-N,N-diethyl-2-cyano-3-(3,4-dihydroxy-5-nitrophenyl) acrylamide of formula (V) is prepared by recrystallizing crude Entacapone from lower aliphatic carboxylic acids such as formic acid or acetic acid with a catalytic amount of hydrochloric or hydrobromic acid as shown below in Scheme-II

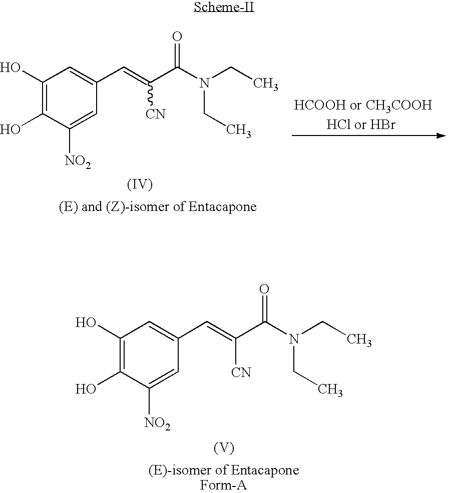

However, this process of isomerization using HBr/Acetic acid suffers with major drawback of operation difficulty as it requires specifically designed glass reactor because of the use of corrosive material. Moreover, it also involves high degree of temperature in highly acidic medium. Further, because of the low reaction volume, it is operationally difficult to transfer the final compound from the reactor.

In summary, process disclosed in prior art for the preparation of (E)-N,N-diethyl-2-cyano-3-(3,4-dihydroxy-5-nitrophenyl)acrylamide, are tedious, time consuming and operationally difficult at industrial scale. Moreover, (E)-N,N-diethyl-2-cyano-3-(3,4-dihydroxy-5-nitrophenyl)acrylamide obtained by prior art process, involves the formation of (Z)-isomer, which causes low yield and affects the purity of the final product.

Therefore, there is a need to develop a process which provides (E)-N,N-diethyl-2-cyano-3-(3,4-dihydroxy-5-nitrophenyl)acrylamide, which is operationally simple at an industrial scale and provides high yield and purity of final product.

With an objective of providing an improved process for the preparation of (E)-N,N-diethyl-2-cyano-3-(3,4-dihydroxy-5-nitrophenyl)acrylamide of formula (I), the present inventors has directed the research work towards developing a process for preparing of (E)-N,N-diethyl-2-cyano-3-(3,4-dihydroxy-5-nitrophenyl)acrylamide of formula (I) which devoid the drawback of the prior art.

Surprisingly, when present inventors had carried out the condensation of 3,4-dihydroxy-5-nitrobenzaldehyde of formula (II) and N,N-diethylcyanoacetamide of formula (III) in piperidine acetate in solvent toluene or ethylacetate to obtain crude Entacapone i.e. N,N-diethyl-2-cyano-3-(3,4-dihydroxy-5-nitrophenyl)acrylamide of formula (IV) with high yield. Moreover, when the crude Entacapone is recrystallized from solvent ethylacetate/methanol in the presence of catalytic amount of iodine, the (E)-N,N-diethyl-2-cyano-3-(3,4-dihydroxy-5-nitrophenyl)acrylamide of formula (I), is obtained in good yield and purity.

Moreover, this makes the process for the preparation of Entacapone operationally simple and easily applicable at an industrial scale.

### OBJECT OF THE INVENTION

Therefore, it is an object of the invention is to provide improved process for the preparation of preparation of (E)-N,N-diethyl-2-cyano-3-(3,4-dihydroxy-5-nitrophenyl) acrylamide of formula (I).

Another object of the invention is to provide an improved process for the preparation of (E)-N,N-diethyl-2-cyano-3-(3,4-dihydroxy-5-nitrophenyl)acrylamide of formula (I) which is operationally simple, cost-effective, easy to handle and feasible at commercial scale.

Yet another object of the present invention is to provide an improved process for the preparation of (E)-N,N-diethyl-2-cyano-3-(3,4-dihydroxy-5-nitrophenyl)acrylamide formula (I) 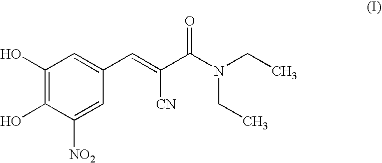
comprising steps of,
(a) condensation of 3,4-dihydroxy-5-nitrobenzaldehyde of formula (II) 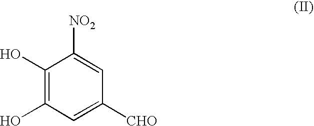
with N,N-diethylcyanoacetamide of formula (III) 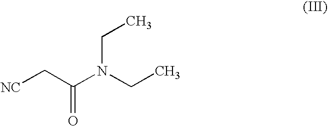
in the presence of a catalyst and optionally in the presence of phase transfer catalyst in a solvent selected from the group comprising of ethylacetate, acetonitrile, hydrocarbon such as toluene, xylene and like or mixture thereof to give mixture of (E) and (Z)-isomer of N, N-diethyl-2-cyano-3-(3,4-dihydroxy-5-nitrophenyl)acrylamide of formula (IV). 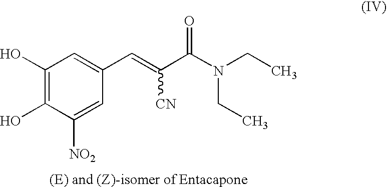
(b) treating an isomeric mixture of (E) and (Z)-isomer of N,N-diethyl-2-cyano-3-(3,4-dihydroxy-5-nitrophenyl)acrylamide of formula (IV) obtained in step (a) with a halogen in catalytic amounts, in a solvent to give (E)-N,N-diethyl-2-cyano-3-(3,4-dihydroxy-5-nitrophenyl)acrylamide formula (I).

### DETAILED DESCRIPTION OF THE INVENTION

Accordingly, the present invention relates to an improved process for the preparation of (E)-N,N-diethyl-2-cyano-3-(3,4-dihydroxy-5-nitrophenyl)acrylamide formula (I) 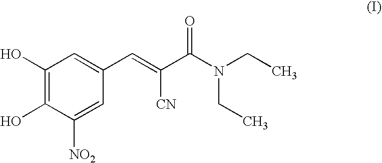
comprising steps of,
(a) condensation of 3,4-dihydroxy-5-nitrobenzaldehyde of formula (II) 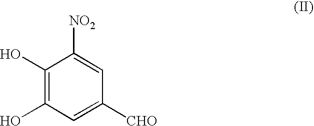
with N,N-diethylcyanoacetamide of formula (111) 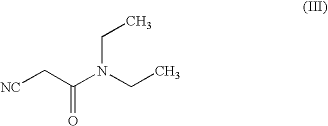
in the presence of a catalyst and optionally in the presence of phase transfer catalyst in a solvent selected from the group comprising of ethylacetate, acetonitrile, hydrocarbon such as toluene, xylene and like or mixture thereof to give mixture of (E) and (Z)-isomer of N, N-diethyl-2-cyano-3-(3,4-dihydroxy-5-nitrophenyl)acrylamide of formula (IV). 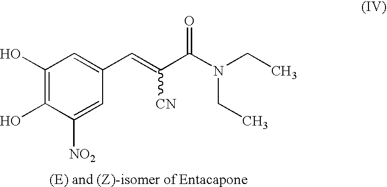
b) treating an isomeric mixture of (E) and (Z)-isomer of N,N-diethyl-2-cyano-3-(3,4-dihydroxy-5-nitrophenyl)acrylamide of formula (IV) obtained in step (a) with a halogen in catalytic amounts, in a solvent to give (E)-N,N-diethyl-2-cyano-3-(3,4-dihydroxy-5-nitrophenyl)acrylamide formula (I).

The example of the suitable catalyst as mentioned hereinabove in step (a) includes but not limited to inorganic base and organic base or salt thereof.

The examples of the base mentioned hereinabove include but not limited to piperidine, pyridine, N-methylmorpholine, morpholine, piperazine and the like or mixture thereof.

The examples of the inorganic and organic salt of base mentioned hereinabove include but not limited sodium acetate, potassium t-butoxide, cesium t-butoxide, peperidinium acetate, pyridine acetate, piperidiniumpropionate and pyridinium para toluene sulfonate and the like or mixture thereof.

The examples of the phase transfer catalyst mentioned hereinabove in step (a) include but not limited to tetrabutylammonium bromide (TBAB), tetrabutylammonium hydroxide, TEBA, tricaprylylmethylammonium chloride, dodecyl sulfate sodium salt, tetrabutylammonium hydrogensulfate, hexadecyl tributyl phosphonium bromide, or hexadecyl trimethyl ammonium bromide.

The conversion of an isomeric mixture of (E) and (Z)-isomer of N,N-diethyl-2-cyano-3-(3,4-dihydroxy-5-nitrophenyl)acrylamide of formula (IV) obtained in step (a) into (E)-N,N-diethyl-2-cyano-3-(3,4-dihydroxy-5-nitrophenyl)acrylamide formula (I) is carried out in the presence of a halogen in catalytic amounts, in a solvent.

The example of halogen mentioned hereinabove in step (b) is selected from the group comprising of bromide, chlorine or iodine and the like.

The examples of the solvent mentioned hereinabove in step (b) include but not limited to ethers such as dioxane, tetrahydrofuran, ethylene glycol, dimethyl ether and the like or mixture thereof; aromatic hydrocarbons such as toluene, xylene and the like or mixture thereof; lower alcohols such as methanol, ethanol, isopropanol, glycols and the like or mixture thereof; polar solvents such as dimethylformamide (DMF), dimethyl sulfoxide (DMSO), acetonitrile, dimethylacetamide; esters such as methylacetete, ethylacetate, butylacetate, t-butylacetate; halogenated hydrocarbons such as methylene dichloride, ethylene dichloride; ketones such as acetone, methyl isobutyl ketone, methylethyl ketone and the like or mixture thereof.

After completion of the reaction, product can be isolated by filtration from reaction mixture and subsequently washing with sodium thiosulfate and water. It can be further purified by recrystallization with acetone, alcohol, ester or hydrocarbon or mixture thereof and product is dried to obtain (E)-N,N-diethyl-2-cyano-3-(3,4-dihydroxy-5-nitrophenyl)acrylamide.

The present invention provides process of preparation of (E)-N,N-diethyl-2-cyano-3-(3,4-dihydroxy-5-nitrophenyl)acrylamide formula (I), which is simple, environment friendly, economical and leads to an enhanced purity.

The process of the present invention has following advantages:
It provides a process which is economical, operational on and industrially applicable.
The process does not involve the use of corrosive material.
The process is simple and easy to handle and does not require special handling care or critical temperature conditions.
It eliminates the use of HBr which is harmful for health.
It reduces the period of time in reaction.
It does not require any specifically designed reactor.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description. Such modifications are intended to fall within the scope of the appended claims.

All patents, patent applications, and publications cited above are incorporated herein by reference in their entirety.

The process of the present invention is described by the following examples, which are illustrative only and should not be construed so as to limit the scope of the invention in any manner.

### EXAMPLE 1

Preparation of Entacapone

A mixture of 3,4-dihydroxy-5-nitrobenzaldehyde (100 gm), N,N-diethyl-2-cyanoacetamide (92.0 gm), piperidine (18.58 gm), acetic acid (13.0 gm) at 20-25° C. in toluene (1000 ml) was refluxed to remove water azeotrophically. After completion of reaction, acetonitrile (1000 ml) was added to clear the reaction mass and charcoalization treatment has been given. Solvent was evaporated and ethyl acetate (1200 ml) was added in the residue then iodine (5 gm) was added to it. Reactions mass was refluxed for 10-12 hrs and cooled to room temperature. Ethyl acetate (2500 ml) was added to reaction mass followed by addition of methanol (1050 ml). Organic layer washed with 4% Sodium thiosulphate solution (600 ml) and water (500 ml). Solvent was evaporated under vacuum till 250 ml of volume remaining in reaction mixture. n-Hexane (1500 ml) was added to the reaction mass and stirred 2 hrs at room temperature. The product filtered and washed with n-hexane (2×150 ml). The product was dried in vacuum to obtain E-isomer of Entacapone.

Yield: 72.28%

Purity: 99%

### EXAMPLE 2

Preparation of Entacapone

A mixture of 3,4-dihydroxy-5-nitrobenzaldehyde (100 gm), N,N-diethyl-2-cyanoacetamide (92.0 gm), piperidine (18.58 g) acetic acid (13.0 g) at 20-25° C. in ethylacetate (1000 ml) was refluxed to remove water azeotrophically. After completion of reaction, iodine (5 gm) was added to it and further stirred the reaction mixture for another 10-12 hrs. The reaction mass is concentrated and cooled and filtered the material. The wet material was suspended in sodium thiosulfate solution in water and filtered, washed with water to get the wet material. Finally it was dried to get the E-isomer of Entacapone.

Yield: 84%

Purity: 99%

1. A process for the preparation of (E)-N,N-diethyl-2-cyano-3-(3,4-dihydroxy-5-nitrophenyl)acrylamide formula (I) 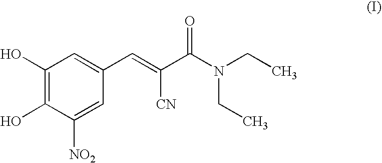
comprising steps of,
(a) condensation of 3,4-dihydroxy-5-nitrobenzaldehyde of formula (II) 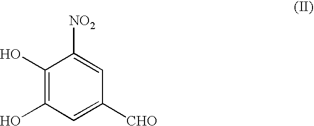
with N,N-diethylcyanoacetamide of formula (III) 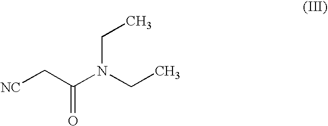
in the presence of a catalyst and optionally in the presence of phase transfer catalyst in a solvent selected from the group comprising of ethylacetate, acetonitrile, toluene, xylene or mixture thereof to give mixture of (E) and (Z)-isomer of N,N-diethyl-2-cyano-3-(3,4-dihydroxy-5-nitrophenyl)acrylamide of formula (IV). 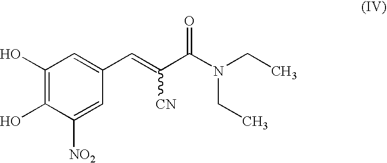
b) treating an isomeric mixture of (E) and (Z)-isomer of N,N-diethyl-2-cyano-3-(3,4-dihydroxy-5-nitrophenyl)acrylamide of formula (IV) obtained in step (a) with a halogen in catalytic amounts, in a solvent to give (E)-N,N-diethyl-2-cyano-3-(3,4-dihydroxy-5-nitrophenyl)acrylamide formula (I).

2. A process according to claim 1, wherein the said catalyst in step (a) is selected from the group comprising of organic and inorganic base or salt thereof.

3. A process according to claim 2, wherein the said base is selected from the group comprising of
piperidine, pyridine, N-methylmorpholine, morpholine, piperazine and the like or mixture thereof.

4. A process according to claim 2, wherein the said organic or inorganic salt of base is selected from the group comprising of sodium acetate, potassium t-butoxide, cesium t-butoxide, peperidinium acetate, pyridine acetate, piperidiniumpropionate and pyridinium para toluene sulfonate or mixture thereof.

5. A process according to claim 1, wherein the said phase transfer catalyst in step (a) is selected from the group comprising of tetrabutylammonium bromide (TBAB), tetrabutylammonium hydroxide, TEBA, tricaprylylmethylammonium chloride, dodecyl sulfate sodium salt, tetrabutylammonium hydrogensulfate, hexadecyl tributyl phosphonium bromide, or hexadecyl trimethyl ammonium bromide.

6. A process according to claim 1, wherein the said solvent in step (b) is selected from a group comprising of dioxane, tetrahydrofuran, ethylene glycol, dimethyl ether, toluene, xylene, methanol, ethanol, isopropanol, dimethylformamide (DMF), dimethylsulfoxide (DMSO), acetonitrile, dimethylacetamide, methylene dichloride, ethylene dichloride, methylacetete, ethylacetate, butylacetate, t-butylacetate; acetone, methyl isobutyl ketone, methylethyl ketone or mixture thereof.

7. A process according to claim 1, wherein the said halogen in step (b) is selected from the group comprising of bromide, chlorine or iodine.

8. A process for the preparation of mixture of (E) and (Z)-isomer of N,N-diethyl-2-cyano-3-(3,4-dihydroxy-5-nitrophenyl)acrylamide of formula (IV) 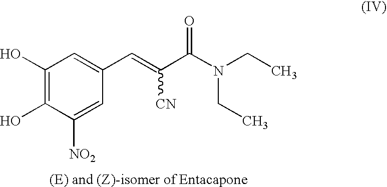
comprising a step of, condensation of 3,4-dihydroxy-5-nitrobenzaldehyde of formula (II) 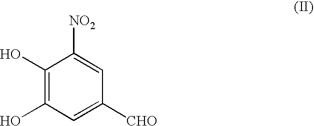
with N,N-diethylcyanoacetamide of formula (III) 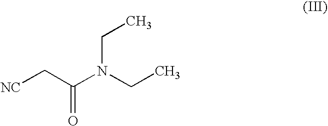
in the presence of a catalyst and optionally in the presence of phase transfer catalyst in a solvent selected from the group comprising of ethylacetate, acetonitrile, hydrocarbon such as toluene, xylene or mixture thereof.

9. A process according to claim 8, wherein the said catalyst is selected from the group comprising of organic and inorganic base or salt thereof.

10. A process according to claim 9, wherein the said base is selected from the group comprising of piperidine, pyridine, N-methylmorpholine, morpholine, piperazine and the like or mixture thereof.

11. A process according to claim 9, wherein the said organic or inorganic salt of base is selected from the group comprising of sodium acetate, potassium t-butoxide, cesium t-butoxide, peperidinium acetate, pyridine acetate, piperidiniumpropionate and pyridinium para toluene sulfonate or mixture thereof.

12. A process according to claim 9, wherein the said phase transfer catalyst is selected from the group comprising of tetrabutylammonium bromide (TBAB), tetrabutylammonium hydroxide, TEBA, tricaprylylmethylammonium chloride, dodecyl sulfate sodium salt, tetrabutylammonium hydrogensulfate, hexadecyl tributyl phosphonium bromide, or hexadecyl trimethyl ammonium bromide.

13. A process for the preparation of (E)-N,N-diethyl-2-cyano-3-(3,4-dihydroxy-5-nitrophenyl)acrylamide formula (I) 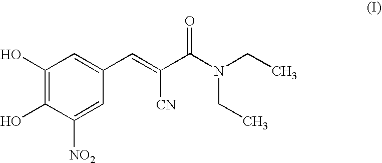
comprising a step of, treating an isomeric mixture of (E) and (Z)-isomer of N,N-diethyl-2-cyano-3-(3,4-dihydroxy-5-nitrophenyl)acrylamide of formula (IV) obtained in step (a) 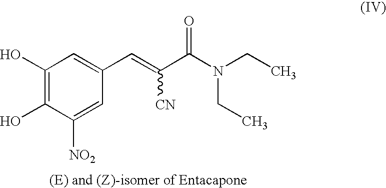
with a halogen in catalytic amounts, in a solvent.

14. A process according to claim 13, wherein the said solvent is selected from a group comprising of dioxane, tetrahydrofuran, ethylene glycol, dimethyl ether, toluene, xylene, methanol, ethanol, isopropanol, dimethylformamide (DMF), dimethylsulfoxide (DMSO), acetonitrile, dimethylacetamide, methylene dichloride, ethylene dichloride, methylacetete, ethylacetate, butylacetate, t-butylacetate; acetone, methyl isobutyl ketone, methylethyl ketone or mixture thereof.

15. A process according to claim 13, wherein the said halogen is selected from the group comprising of bromide, chlorine or iodine.
